(19) United States
(12) Patent Application Publication
(10) Pub. No.: US 2017/0119680 A1
(43) Pub. Date: May 4, 2017

(54) EXTENDED RELEASE FILM-COATED CAPSULES

(71) Applicant: R.P. SCHERER TECHNOLOGIES, LLC, CARSON CITY, NV (US)

(72) Inventors: IRENA MCGUFFY, LUTZ, FL (US); WILLIAM M. BELL, LUTZ, FL (US)

(21) Appl. No.: 14/928,029

(22) Filed: Oct. 30, 2015

(51) Int. Cl. A61K 9/48; A61K 47/32; A61K 47/38; A61K 47/34; A61K 31/192; A61K 31/445

(52) U.S. Cl. CPC A61K 9/4891; A61K 31/192; A61K 31/445; A61K 9/4833; A61K 9/4825; A61K 47/38; A61K 47/34; A61K 47/32

(57) ABSTRACT

Pharmaceutical formulations, preferably in the form of softgel capsules or hard-shell capsules, exhibit extended release through the use of a coating comprising a water-insoluble polymer and a pH-independent pore former. Extended release from softgel capsules and hard-shell capsules can be achieved without the use of lipid-based semi-solid or solid materials.

### TECHNICAL FIELD

This invention relates to extended release pharmaceutical formulations, preferably in the form of softgel capsules or hard-shell capsules, that substantially extend the release of drugs into the gastrointestinal (“GI”) tract, resulting in lower Cₘₐₓ, extended drug effects, and potentially reduced side effects. This invention also relates to processes for the preparation of the extended release pharmaceutical formulations.

### BACKGROUND

Oral drug delivery typically requires drug products to release drug molecules to form a solution in the GI tract so the drug can be absorbed across the gut wall and enter systemic circulation. For reasons of product efficacy and safety, drug molecule release may need to take place in a controlled manner with a release profile that meets the therapeutic requirements of the product. Mechanisms of controlled release include delayed release, pulsatile release and extended release. These mechanisms have inherent pharmacokinetic differences and result in drug products that may not be bioequivalent with regard to the same pharmaceutical active ingredient in the same strength. Delayed release may refer to enteric-coated products that delay release of the drug molecules until the product passes through the stomach, thereby preventing damage to the drug from stomach acidity, after which the drug is released via immediate-release in the GI tract or via timed release where the drug is released at some point in the GI tract, but typically after passing through the stomach. Delayed release products may require enteric, pH-dependent coatings that are stable at the highly acidic pH found in the stomach but break down rapidly at a less acidic pH in the GI tract, thereby enabling immediate-release of the drug in lower portions of the GI tract, or pH-independent coatings combined with pH-dependent pore formers, which allow for drug release in lower portions of the GI tract. On the other hand, extended release products which are not enteric-coated, are formulated in such a manner as to make the drug available over an extended period of time following ingestion. In the extended release mechanism, the drug is uniformly released over a desired, extended period of time even if the drug is formulated in an immediate-release fill. Extended release provides several advantages, including reduced dose frequency, resulting in improved patient compliance, potential attenuation of adverse side effects, and increased duration of drug therapeutic effect.

The extended release mechanism of drugs in oral solid dosage forms is typically achieved by formulating the dosage forms where the active ingredient is embedded in a semi-solid or solid matrix composed of a mixture of lipid-based semi-solid or solid materials. Such materials include hydrophilic and hydrophobic materials including waxes, long chain fatty acids (e.g., stearic acid), long chain alcohols (e.g., cetyl alcohol and cetostearyl alcohol), long chain fatty acid glyceryl esters (e.g., glyceryl behenate, glyceryl distearate, glyceryl palmitosearate, sucrose esters, polyoxyl glycerides, etc.), high molecular weight hydrophilic polymers (e.g., hypromellose, hydroxypropyl cellulose, hydroxyethyl cellulose, polycarbophil, polyvinyl alcohol, etc.) and/or water-insoluble polymers (e.g., ethyl cellulose, cellulose acetate phthalate, polyvinyl acetate, polyethylene oxide, etc.). The solid dosage form may also be coated using various pharmaceutically acceptable polymers. However, a number of such lipid-based semi-solid or solid materials are of natural origin and can undesirably have variable composition and performance characteristics. In addition, a number of such lipid-based semi-solid or solid materials can undergo crystalline form changes at various storage conditions, thereby affecting drug stability and resulting in a change in drug release profile.

While the extended release mechanism of drugs in oral solid dosage forms can be achieved in tablets, pellets, or hard-shell capsules, the release of poorly soluble compounds from these semi-solid or solid matrices may be non-uniform and unpredictable, resulting in high inter-patient and intra-patient variability. Hard-shell capsules and softgel capsules offer the additional possibility of using a liquid, solution, suspension, or emulsion in a solid oral dosage form. Hard-shell capsules and softgel capsules, then, offer the flexibility for delivering poorly soluble drugs as solutions, suspensions, or emulsions, leading to improved absorption of these drugs compared to delivery from a tablet or pellet form.

Despite this flexibility over tablets and pellets, formulation challenges exist for extended release gelatin-based softgel capsules and hard-shell capsules due to the lipid-based matrices of semi-solid or solid materials. In particular, these formulations with semi-solid or solid lipid-based fill systems have higher melting points and thus do not lend themselves readily to encapsulation using conventional gelatin-based encapsulation films, since during capsule formation the films have sealing temperature limits that are lower than the higher melting points of these semi-solid or solid lipid-based fill systems. Accordingly, formulation of extended release softgel capsules and hard-shell capsules typically necessitates the use of technology (e.g., Catalent Pharma Solutions' OptiShell™ technology) that provides for polysaccharide-based, non-gelatin shells for encapsulating high melting point lipid-based semi-solid or solid fill formulations. However, despite the option of using technology such as OptiShell™, some drugs may be sensitive to elevated temperature, incompatible with, and/or insoluble in a semi-solid or solid lipid-based matrix. Thus, it is desirable to achieve extended release of drugs in oral solid dosage forms without the use of semi-solid or solid lipid-based matrices.

Accordingly, there is currently a need in the state of the art for softgel and hard-shell extended release formulations for the delivery of pharmaceutically active ingredients that avoid the use of lipid-based semi-solid and solid matrices that can cause crystalline form changes at various storage conditions, affect drug stability, result in a change in drug release profile, require elevated temperature during processing, and be incompatible with certain active ingredients. There is also a need in the state of the art for developing extended release softgel and hard-shell capsules that allow for the use of conventional softgel capsules (e.g., gelatin-based) and hard-shell capsules (e.g., gelatin- and hypromellose-based). The present invention satisfies these needs with novel softgel and hard-shell capsules that exhibit extended release through use of a film coating comprising a water-insoluble polymer and a pH-independent pore former, thereby achieving extended release without the conventional use of semi-solid and solid lipid-based matrix systems. While this approach has been attempted for coating tablets, it has never been used for the development of extended release softgel capsules and hard-shell capsules and, in particular, for the development of extended release softgel capsules and hard-shell capsules for the delivery of a liquid or semi-solid immediate-release fill. Using such an approach with softgel and hard-shell capsules could potentially create safety risks given the possibility of coating failures, dose dumping, etc. The present invention provides for extended release formulations that not only allow for the use of polysaccharide-based (e.g., non-gelatin) softgel and hard-shell capsules, but also allow for the use of conventional softgel capsules (e.g., gelatin-based) and hard-shell capsules (e.g., gelatin- and hypromellose-based) for the extended delivery of liquid or semi-solid immediate-release fills.

U.S. Patent Application Publication No. 2005/0244489 A1 describes liquid compositions for soft, sustained-release capsules and methods for their production. This publication describes fill formulations that gel in situ after encapsulation, thereby forming a sustained release matrix. In contrast, the formulations of the present invention are not subject to post-encapsulation gelling and do not form a matrix.

European Patent No. 0173293 B1 describes sustained release terfenadine formulations in the form of hard or soft shelled gelatin capsules. However, in contrast to the present invention, the sustained release formulations of this publication contain a solid mass/matrix fill.

WO 2002/087543 A1 and U.S. Patent Application Publication No. 2004/0253306 A1 describe sustained release formulations of nifedipine and dextromethorphan that are compatible with a soft elastic gelatin capsule and a two-piece hard-shell gelatin capsule. However, in contrast to the present invention, the sustained release formulations of these publications are directed to fills that spontaneously form liposomes upon introduction to the aqueous environment.

WO 2007/044488 A1, U.S. Patent Application Publication No. 2005/0220878 A1, U.S. Patent Application Publication No. 2002/0114832 A1, and U.S. Patent Application Publication No. 2009/0136650 A1 disclose softgel capsules. However, in contrast to the present invention, these publications relate to delayed release, enteric-coated formulations.

European Patent No. 1128821 B1, WO 2000/035419 A2, U.S. Pat. No. 6,419,952 B2, Australian Patent No. 765909 B2, European Patent No. 1140012 B1, U.S. Pat. No. 6,183,845, and U.S. Pat. No. 6,929,803 describe multi-layer softgel shells or softgel capsules with multiple coating layers. In contrast, certain embodiments of the present invention are directed to single-layer capsules that require only one coating layer.

U.S. Patent Application Publication No. 2010/0087520 A1 and WO 2010/042499 A1 describe liquid orlistat-containing fill materials suitable for encapsulating in hard or soft capsules. However, in contrast to the present invention, rate-controlling polymers in these publications are incorporated in the shell mass.

U.S. Pat. No. 5,300,300 and European Patent No. 0508312 B1 describe controlled release pharmaceutical formulations for oral administration coated by an enterosoluble gastroresistant film and containing bile acids and their salts. However, in contrast to the present invention, these publications are directed to pH-dependent coatings and formulations that consist of non-coated portions.

U.S. Patent Application Publication No. 2012/0244216 A1 and European Patent No. 2081550 A2 describe coated pharmaceutical capsule dosage forms wherein the coatings contain the pharmaceutically active ingredients. In contrast, the present invention is not directed to coating formulations with the active ingredient.

U.S. Pat. No. 5,120,548 describes a controlled release drug delivery device that is based primarily on swellable polymers and is directed to tablets or ocular inserts. In contrast, the present invention is directed to water-insoluble polymers and softgel and hard-shell capsules.

U.S. Pat. No. 7,790,215 B2 describes controlled release powder-filled capsules and tablets that are coated with a mixture of gelatin and hydrophobic polymer. In contrast, the present invention is directed to softgel and hard-shell capsules.

U.S. Patent Application Publication No. 2004/0063784 A1 and U.S. Pat. No. 6,849,661 B2 describe the use of verapamil to reduce abnormal gastrointestinal motility. Though it is mentioned that this could be achieved by formulating verapamil in a liquid formulation, which may be filled into soft gelatin capsules, no guidance is provided in doing so.

U.S. Patent Application Publication No. 2010/0278917 A1 describes methods and formulations for treating inflammatory bowel disease which include 4- and/or 5-aminosalicylic acid and modified release dosage forms. However, this publication is directed to matrix tablets, while the present invention is focused on a combination of water-insoluble polymers with water-soluble pore formers applied onto the surface of softgel or hard-shell capsules.

U.S. Patent Application Publication No. 2009/0220613 A1 describes coated delivery devices for controlled release of active ingredient. However, this publication is directed to tablets and pellets, while the substrates for the coating systems of the present invention are liquid or semi-solid-filled softgel or hard-shell capsules.

U.S. Patent Application Publication No. 2009/0017110 A1 describes formulations containing mesalamine as the active ingredient. Furthermore, mesalamine was formulated into beads (pellets) or tablets, not softgel or hard-shell capsules filled with liquid or semi-solid material, as seen in the present invention. Lastly, water-swellable polymers described in this publication are pH-dependent polymers, while the present invention focuses on pH-independent pore formers.

U.S. Patent Application Publication No. 2011/0287093 A1 describes a controlled release core and immediate-release gelatin capsule around it. In contrast, the present invention focuses on the exact opposite—immediate-release core and controlled release coating around the capsule.

U.S. Patent Application Publication No. 2002/0155154 A1 and U.S. Pat. No. 6,929,803 B2 describe a gelatin capsule containing a liquid formulation and coated with multiple layers and having an exit orifice through which the fill contents are released. Though these publications describe several ways in which the exit orifice can be formed, such as through mechanical drilling, laser drilling, or leaching a passageway former from the composite wall, these publications fail to disclose the use of pore formers as seen in the present invention. In contrast to these publications, the present invention achieves a controlled release profile without the need for multiple layers and does not require mechanical or laser drilling. Furthermore, in contrast to these publications, the release is achieved through a plurality of orifices, which are formed after the pore former is dissolved.

U.S. Patent Application Publication No. 2005/0152967 A1 describes a combination of two drugs: expectorant (with immediate-release profile) and decongestant (with extended release profile). Extended release of the decongestant is achieved by coating drug-loaded beads and filling them into hard-shell capsules or making effervescent tablets. The drugs are in solid form and release profile is modified before encapsulation, not after encapsulation. In contrast, in the present invention, the drug is dissolved or suspended in the liquid or semi-solid immediate-release fill material and the capsules are coated to achieve extended release.

By providing for novel softgel and hard-shell capsules that exhibit extended release through use of a film coating comprising a water-insoluble polymer and a pH-independent pore former, the present invention advances the state of the art.

### SUMMARY OF THE INVENTION

The present invention is directed to an extended release oral solid dosage form comprising: (a) a fill material, said fill material comprising a liquid or semi-solid fill material containing at least one pharmaceutically active ingredient; (b) a capsule, said capsule comprising a gelatin- or non-gelatin based softgel capsule or a hard-shell capsule, containing the fill material; and (c) a coating surrounding the capsule, said coating comprising (1) a water-insoluble polymer and (2) a pore former. In a preferred embodiment, the liquid or semi-solid fill material is an immediate-release fill material.

In certain preferred embodiments of the invention, the water-insoluble polymer is a pharmaceutically acceptable polymeric material having low solubility in the different pHs of the stomach and GI tract, i.e., having low solubility in a pH range of about 1 to 8. In additional preferred embodiments of the invention, the pore former is comprised of a water-soluble, pH-independent material. In further embodiments, the film coating composition further comprises plasticizers, surfactants, detackifying agents, antifoaming agents, colorants, opacifiers, and/or combinations thereof.

The film coating compositions of the present invention function to provide extended or, preferably, zero-order release of a pharmaceutically active ingredient by forming a barrier around the capsules and allowing the fill materials and pharmaceutically active ingredient to escape through small openings (i.e., pores) in the water-insoluble polymer created by the pore former.

The present invention is also directed to a process of preparing the extended release formulations of the present invention.

### BRIEF DESCRIPTION OF THE FIGURES

FIG. 1 and FIG. 2 show the release profiles of individual units of a coated, extended release softgel formulation according to an example embodiment of the present invention after 3 and 17 months' storage, respectively.

FIG. 3 and FIG. 4 show the average release profiles of a coated, extended release softgel formulation according to an example embodiment of the present invention after 3 and 17 months' storage, respectively.

FIG. 5 shows the release profile of an uncoated softgel formulation.

FIG. 6 and FIG. 7 show the release profiles of a coated, extended release softgel formulation according to an example embodiment of the present invention after 1 and 15 months' storage, respectively.

FIGS. 8-13 show the release profiles of coated, extended release softgel formulations according to further example embodiments of the present invention.

FIG. 14 shows the release profile of an uncoated softgel formulation.

### DETAILED DESCRIPTION OF THE INVENTION

The present invention advances the state of the art by developing oral solid dosage forms that achieve extended release of pharmaceutically active ingredients without the use of semi-solid or solid lipid-based matrix systems. In particular, in the present invention, extended release can be achieved in not only polysaccharide-based (e.g., non-gelatin-based) softgel and hard-shell capsules, but also in conventional softgel capsules (e.g., gelatin-based) and hard-shell capsules (e.g., gelatin- and hypromellose-based) with liquid or semi-solid, preferably immediate-release, fills. Extended release is achieved in the present invention by, at least in part, a coating applied to the surface of the oral solid dosage forms.

According to a first embodiment of the invention, an extended release oral solid dosage form comprises: (a) a fill material, said fill material comprising a liquid or semi-solid fill material containing at least one pharmaceutically active ingredient; (b) a capsule, said capsule comprising a softgel capsule or a hard-shell capsule, containing the fill material; and (c) a coating surrounding the capsule, said coating comprising (1) a water-insoluble polymer and (2) a pore former. In a preferred embodiment, the present invention is directed to a single-layer capsule that requires only one coating. In certain preferred embodiments, the coating of the present invention may be applied to, without limitation, round, oval, oblong, and other shaped capsules.

As used with respect to the present invention, the term “oral solid dosage form” includes, without limitation, softgel capsules and hard-shell capsules. As used herein, the terms “softgel capsules” and “hard-shell capsules” include, without limitation, gelatin-free softgel and hard-shell capsules (e.g., polysaccharide, polyvinyl alcohol or other polymer-based capsules) and conventional gelatin-based softgel capsules and gelatin- or hypromellose-based hard-shell capsules. In a preferred embodiment, the coating of the present invention is applied to the surface of conventional gelatin-based softgel capsules and gelatin- or hypromellose-based hard-shell capsules.

The fill material of the present extended release oral solid dosage forms is a liquid or semi-solid fill material; the fill material may be a liquid pharmaceutically active ingredient without any additional excipients. In a preferred embodiment, the fill material is an immediate-release fill material. In a preferred embodiment, the fill material is also hydrophilic. Preferably, the capsules are filled with a water-miscible, dispersible fill material that includes, but is not limited to, one or more of low-HLB surfactants (e.g., glyceryl monooleate (type 40) (Peceol™)), linoleoyl polyoxyl-6 glycerides (e.g., Labrafil® M2125CS), oleoyl polyoxyl-6 glycerides (e.g., Labrafil® M1944CS), lauroyl polyoxyl-6 glycerides (e.g., Labrafil® M2130CS), polyglyceryl-3 dioleate (e.g., Plurol® Oleique CC 497), mono- and diglycerides of caprylic and capric acid (e.g., various grades of Capmul® MCM, Imwitor®, etc.), sorbitan esters of fatty acids (e.g., Span® 20, Span® 80, etc.), or high HLB value surfactants (e.g., caprylocaproyl polyoxyl glycerides, such as Labrasol® and Acconon® MC8-2), polyoxyl 35 castor oil (e.g., Kolliphor® EL), polyoxyl 40 hydrogenated castor oil (e.g., Kolliphor® RH40), vitamin E TPGS, polyethylene glycol-15-hydroxystearate (e.g., Kolliphor® HS 15), lauroyl polyoxyl-32 glycerides (e.g., Gelucire® 44/14), stearoyl polyoxyl-32 glycerides (e.g., Gelucire® 50/13), Gelucire® 48/16, polysorbate 20, polysorbate 40, polysorbate 60, polysorbate 80, etc.) and/or solvents or co-solvents such as polyethylene glycol in a range of molecular weights, triethyl citrate, triacetin, diethylene glycol monoethyl ether (e.g., Transcutol®), free fatty acids (e.g., caprylic acid, lauric acid, oleic acid, linoleic acid, etc.), ethanol, propylene glycol, glycerin, water and combinations thereof that enhance dispersibility of the fill material and reduce capsule-to-capsule release profile variability.

As used herein, “pharmaceutically active ingredient” refers to a drug product that may be used in the diagnosis, cure, mitigation, treatment, or prevention of disease. Any pharmaceutically active ingredient may be used for purposes of the present invention, including both those that are water-soluble and those that are poorly soluble in water. Suitable pharmaceutically active ingredients include, without limitation, analgesics and anti-inflammatory agents, antacids, anthelmintics, anti-arrhythmic agents, anti-bacterial agents, anti-coagulants, anti-depressants, anti-diabetics, anti-diarrheals, anti-epileptics, anti-fungal agents, anti-gout agents, anti-hypertensive agents, anti-malarials, anti-migraine agents, anti-muscarinic agents, anti-neoplastic agents and immunosuppressants, anti-protozoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, oral vaccines, proteins, peptides and recombinant drugs, sex hormones and contraceptives, spermicides, stimulants, and combinations thereof.

In a preferred embodiment, the water-insoluble polymers of the coating of the present invention are pharmaceutically acceptable polymeric materials having low solubility in the different pHs of both the stomach and the lower parts of the GI tract (i.e., small and large intestine). In particular, the polymers are preferred to have low solubility in the pH range of about 1 to about 8. As used herein, “low solubility” or “slightly soluble” refers to 0.0001 M to 0.1 M at room temperature, while “insoluble” or “sparingly soluble” refers to less than 0.0001 M at room temperature or to a substance of which less than 0.1 g dissolves in 100 mL solvent at room temperature. Suitable water-insoluble polymers include, without limitation, ethylcellulose (e.g., Ethocel, Aquacoat ECD, Surelease®), ethyl acrylate and methylacrylate copolymer (e.g., Eudragit NE30D), polyvinyl acetate (e.g., Kollidon DR and Kollicoat SR 30D), cellulose acetate, etc. Careful selection of the water-insoluble polymer is critical and required in order to achieve acceptable film adhesion to softgel and hard-shell capsules. The amount of water-insoluble polymer in the coating composition ranges preferably from about 1% to about 30%, more preferably from about 5% to about 20%, and most preferably from about 5% to about 15% of the total dry polymer weight. As used herein, “total dry polymer weight” refers to the total weight of polymer applied to the capsule when the amount of water originally present in the aqueous suspension is removed.

In a preferred embodiment, the pore formers of the coating of the present invention are water-soluble, pH-independent materials. Suitable pore formers include, without limitation, hypromellose (e.g., Methocel and Pharmacoat), hydroxypropyl cellulose, hydroxyethyl cellulose, methyl cellulose, polyvinyl alcohol polyethylene glycol graft copolymer (e.g., Kollicoat IR), povidone, sucrose, water-soluble sodium and potassium salts, gelatin, cyclodextrins, copovidone, dextrates, dextrose, lactitol, mannitol, erythritol, fructose, galactose, lactose, hydroxyethyl methylcellulose, maltodextrin, maltose, sorbitol, propylene glycol, xylitol, tagatose, trehalose, polyethylene glycols, poloxamers, polydextrose, polyvinyl alcohol, etc. Careful selection of the pore former is critical and required in order to achieve adequate release of the fill material from softgel and hard-shell capsules. Desirable pore former properties include sufficient solubility in water, i.e., leaving enough pores to result in release but that doesn't affect the physical integrity of the coating. The amount of pore former in the coating composition ranges preferably from about 1% to about 50%, more preferably from about 3% to about 40%, and most preferably from about 4% to about 30% of the total dry polymer weight.

In certain preferred embodiments, the coating of the present invention further comprises a plasticizer. Suitable plasticizers include, without limitation, triethyl citrate, tributyl citrate, acetyltriethyl citrate, acetyltributyl citrate, triacetin, propylene glycol, poloxamer, polyethylene glycols, dibutyl sebacate, butyl stearate, dibutyl phthalate, diethyl phthalate, dimethyl phthalate, etc. Careful selection of the plasticizer is critical and required since low plasticizer content or poor selection of plasticizer can cause cracking of the coating, while high levels of plasticizers can cause challenges such as capsules sticking during coating and/or storage. Desirable plasticizer properties include the ability to lower Tg and film forming temperature and miscibility with polymer. If included, the amount of plasticizer in the coating composition ranges preferably from about 0% to about 60%, more preferably from about 0% to about 50%, and most preferably from about 0% to about 40% of the total dry polymer weight.

In certain embodiments of the present invention, the coating further comprises a surfactant, a detackifying agent, an antifoaming agent, and/or combinations thereof. Suitable surfactants include, without limitation, high HLB, water-soluble or water-miscible surfactants such as polyoxyethylene alkyl ethers, polyoxyethylene castor oil derivatives, polyoxyethylene sorbitan fatty acid esters, polyoxyethylene fatty acid esters, polyoxylglycerides, vitamin E TPGS, sorbitan fatty acid esters, etc. Suitable detackifying agents include, without limitation, talc, glyceryl fatty acid esters, etc. Suitable antifoaming agents include, without limitation, simethicone, dimethicone, etc. One of ordinary skill in the art would readily appreciate suitable inclusion amounts for each of these additional components.

The amount of coating applied to the capsule can vary depending on the desired effects. For example, if faster release is desired, then a lower coating weight gain is in order; of course, one of ordinary skill in the art would readily understand that considerations regarding how much coating is applied are polymer specific. Generally the amount of coating applied is described in terms of weight gain of the dosage form. According to the invention, coating weight gain preferably ranges from 5% to 100%, more preferably from 5% to 75%, and even more preferably from 5% to 40%.

Though not required, an optional sub-coat may be applied underneath the extended release coating on the capsules in order to improve adhesion of the water-insoluble polymer or uniformity of the extended release film. Similarly, an optional top-coat may be applied on the surface of the extended release coating on the capsules in order to reduce capsule sensitivity to higher ambient moisture level and/or temperature and to reduce/prevent agglomeration. Exemplary sub- and top-coats include, without limitation, hydroxypropylmethylcellulose, polyvinyl alcohol, and aminomethacrylate copolymer-based coats and others that tend to allow less water uptake by the dosage form.

According to a second embodiment of the present invention, an extended release oral solid dosage form is prepared by the process comprising the steps of: (a) preparing a fill material, said fill material comprising a liquid or semi-solid fill material containing at least one pharmaceutically active ingredient; (b) encapsulating the fill material of step (a) with a capsule, said capsule comprising a softgel capsule or a hard-shell capsule; (c) applying a coating onto the surface of the capsule, said coating comprising (1) a water-insoluble polymer and (2) a pore former.

Fill materials may be prepared in any conventional manner. Details regarding components of the fill material including the at least one pharmaceutically active ingredient are the same as set forth above with regard to the first embodiment of the invention. As an example, fill material may be prepared in a closed stainless steel vessel capable of mixing under vacuum or in a suitably sized non-reactive vessel.

Encapsulation of the fill material can be accomplished in any conventional manner. Details regarding the capsule and fill material are the same as set forth above with regard to the first embodiment of the invention. As an example, a rotary die encapsulation process with positive displacement dosing may be used for this purpose.

The coating of the present invention is prepared by dispersing the water-insoluble polymer in water or other aqueous media and dissolving the pore former in the same media. Alternatively, a mixture of water and organic solvent or a solvent-based coating solution/suspension may be used.

The coating can be applied by any conventional means. For example, the coating can be applied by spraying the coating solution/suspension onto the surface of the capsules in a perforated coating pan, semi-perforated coating pan, non-perforated coating pan, sugar coating pan, fluid bed coater/dryer, or any other piece of equipment suitable for film coating. The coating process can be a batch process or a continuous process. With respect to the preferred softgel and hard-shell capsules of the present invention, careful manufacturing is required due to the challenges of sensitivity of the capsules to heat, high spray rates, etc. High product temperature, e.g., in excess of 45°−50° C., can result in capsules melting and agglomerating in the pan resulting in a failed batch. On the other hand, too low of a product temperature, e.g., less than 25° C., during coating may result in inadequate drying/evaporation of water, resulting in capsule agglomeration. In addition, to prevent over-wetting and ensure adequate drying capacity during coating, factors such as spray rate, inlet air temperature, process air volume, atomization and pattern air pressure may be optimized. One of ordinary skill in the art would readily understand how to manipulate the relevant parameters.

The process of the present invention may further comprise the steps of applying a sub-coat prior to applying the coating and/or applying a top-coat after applying the coating.

The novel extended release oral solid dosage forms of the present invention exhibit extended release of the pharmaceutically active ingredient due to the coating. In particular, the water-insoluble polymer forms a barrier and allows the fill material including the pharmaceutically active ingredient to escape through small openings in the polymer formed by the pore formers. The coating of the present invention allows small amounts of GI fluid to migrate through the polymer and slowly partially or complete dissolve the capsule shell. Dissolution of the shell does not result in disintegration of the dosage form; rather it allows the fill material to migrate through the same insoluble polymer and result in extended drug release from the dosage form. The undissolved shell may be broken up by the GI tract after the fill is released or excreted unchanged.

The release profile from the extended release oral solid dosage forms of the present invention may include an initial lag time, during which the pore former dissolves in the media, leaving pores in the coating and allowing dissolution of a portion of or the entire shell. This, in turn, allows for the fill material to travel through the pores in the coating and be released into the media. Extended release of the present invention is achieved due to the combination of the film coating characteristics and the fill material characteristics of the present invention. Surprisingly, due to the combination of the film coating characteristics and the hydrophilic fill material characteristics, unit to unit variability is smaller than would be expected for oil-based coated products.

The rate of release can be modified by modifying pore former type or level or coating weight gain. The pore formers are water-soluble and pH-independent, which results in continuous release of the fill, regardless of the pH.

A further advantage discovered by the inventors is that the release profile of the extended release oral solid dosage forms of the present invention remains essentially unchanged over the product's shelf-life at room temperature for up to preferably 18 months, more preferably at least 2 years. As used herein, “essentially unchanged” refers to less than 20% absolute change in the amount of drug released at each time point.

Specific embodiments of the invention will now be demonstrated by reference to the following examples. It should be understood that these examples are disclosed solely by way of illustrating the invention and should not be taken in any way to limit the scope of the present invention.

### Example 1

Gelatin-based softgel capsule formulations of 200 mg Ibuprofen were prepared according to the composition set forth in Table 1.

TABLE 1
Composition of 200 mg Ibuprofen Softgel Capsule.

| | % w/w | mg/capsule |
|---|---|---|
| Ibuprofen | 36.8 | 200 |
| Polyethylene Glycol 600 | 21.6 | 117 |
| Potassium Hydroxide | 4.6 | 25 |
| Purified Water | 3.3 | 18 |
| Gelatin | 21.4 | 116 |
| Sorbitol, liquid | 12.2 | 66 |
| FD&C Blue #1 | 0.007 | 0.04 |
| Total | 100 | 542 |

The capsules were then coated with the film coating composition set forth in Table 2 in order to produce extended release softgel capsules.

TABLE 2
Coating Composition.

| | Function | % w/w | mg/capsule |
|---|---|---|---|
| Ethylcellulose dispersion | Water-insoluble film- | 71.4 | 67 |
| (Aquacoat ECD 30) | forming polymer | | |
| Triethyl citrate | Plasticizer | 14.3 | 13 |
| Polyvinyl | Water-soluble pore | 14.3 | 13 |
| alcohol/polyethylene | former | | |
| glycol co-polymer | | | |
| (Kollicoat IR) | | | |
| Water | Solvent | N/A | N/A |
| Total | N/A | 100.0 | 93 |

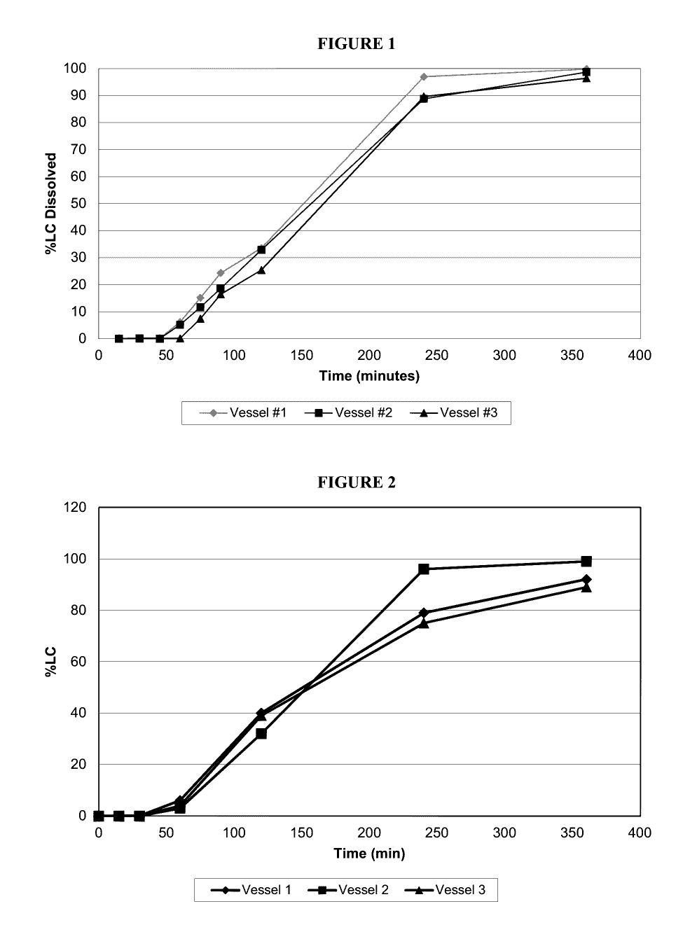

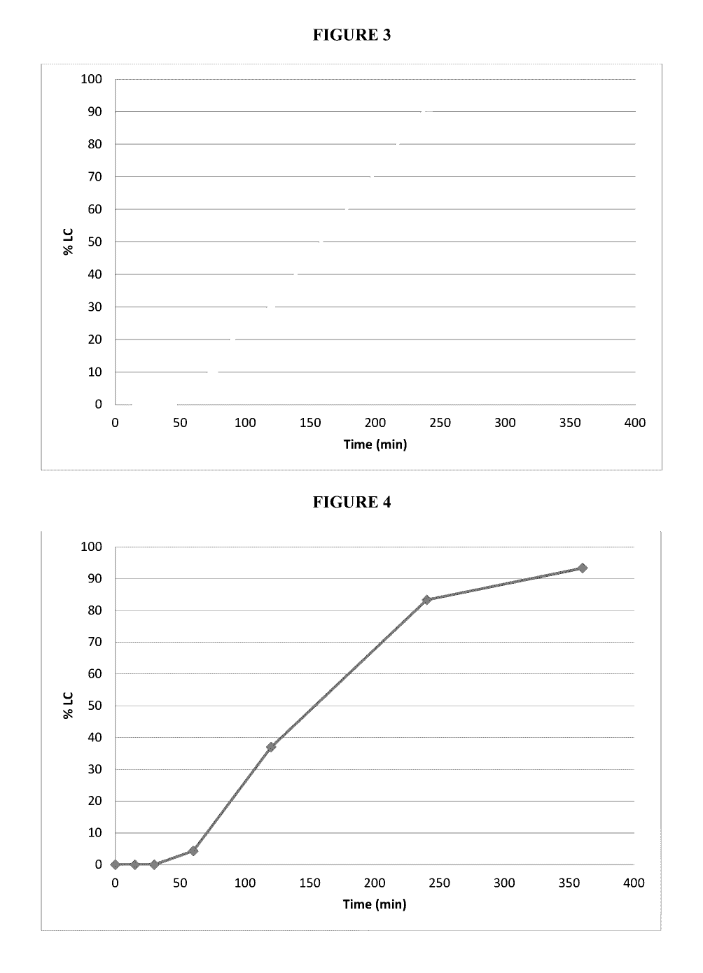

Subsequent experimental tests were run to obtain the release profiles of these capsules. FIG. 1 shows the release profile of three units of the coated capsules according to this example embodiment after 3 months' storage at room temperature. FIG. 2 shows the release profile of the three units of the coated capsules according to this example embodiment after 17 months' storage at room temperature. FIGS. 3 and 4 show the average release profile of the three units of the coated capsules according to this example embodiment after 3 months' storage and 17 months' storage at room temperature, respectively.

As seen in FIGS. 1-4, the coated softgel capsules according to this example embodiment enable extended release or zero-order release, i.e., at a constant rate, of the liquid/semi-solid fill from the capsules without the use of semi-solid or solid lipid-based matrices.

### Comparative Example 1

Gelatin-based oval softgel capsule formulations containing 200 mg Ibuprofen were prepared according to the composition set forth in Table 1. However, the capsules in this comparative example remained uncoated.

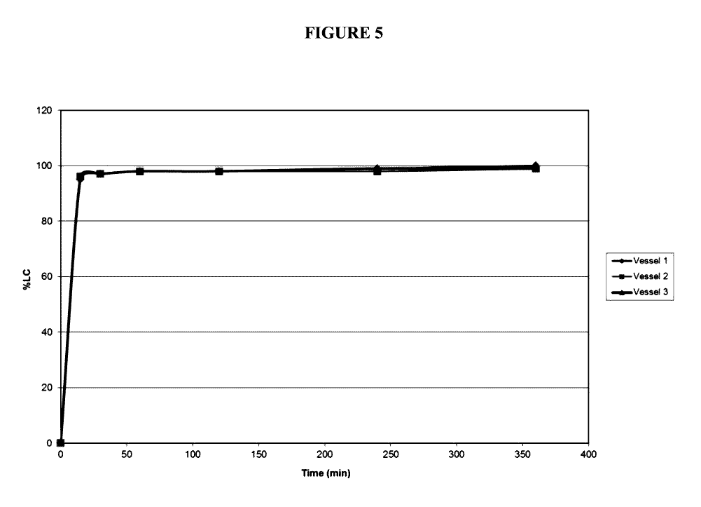

FIG. 5 shows that an uncoated softgel capsule of this comparative example, which is representative of softgel capsules in the state of the art, exhibits immediate-release of the fill material. In contrast, FIGS. 1-4 show that an example embodiment of the present invention enables zero-order or close to zero-order release from softgel capsules.

### Example 2

Gelatin-based softgel capsule formulations containing 200 mg Ibuprofen were prepared according to the composition set forth in Table 1. The capsules were then coated with the film coating composition set forth in Table 3 in order to achieve extended release softgel capsules.

TABLE 3
Coating Composition.

| | Function | % w/w | mg/capsule |
|---|---|---|---|
| Ethyl acrylate and methyl | Water-insoluble film- | 42.86 | 52 |
| methacrylate copolymer | forming polymer | | |
| (Eudragit NE 30D) | | | |
| Hypromellose (Methocel | Water-soluble pore | 4.76 | 6 |
| E3 Premium LV) | former | | |
| Polysorbate 80 (Tween | Surfactant | 4.76 | 6 |
| 80 HP LQ-MH) | | | |
| Talc | Detackifying agent | 47.62 | 58 |
| Water | Solvent | N/A | N/A |
| Water | Solvent | N/A | N/A |
| Total | N/A | 100.0 | 122 |

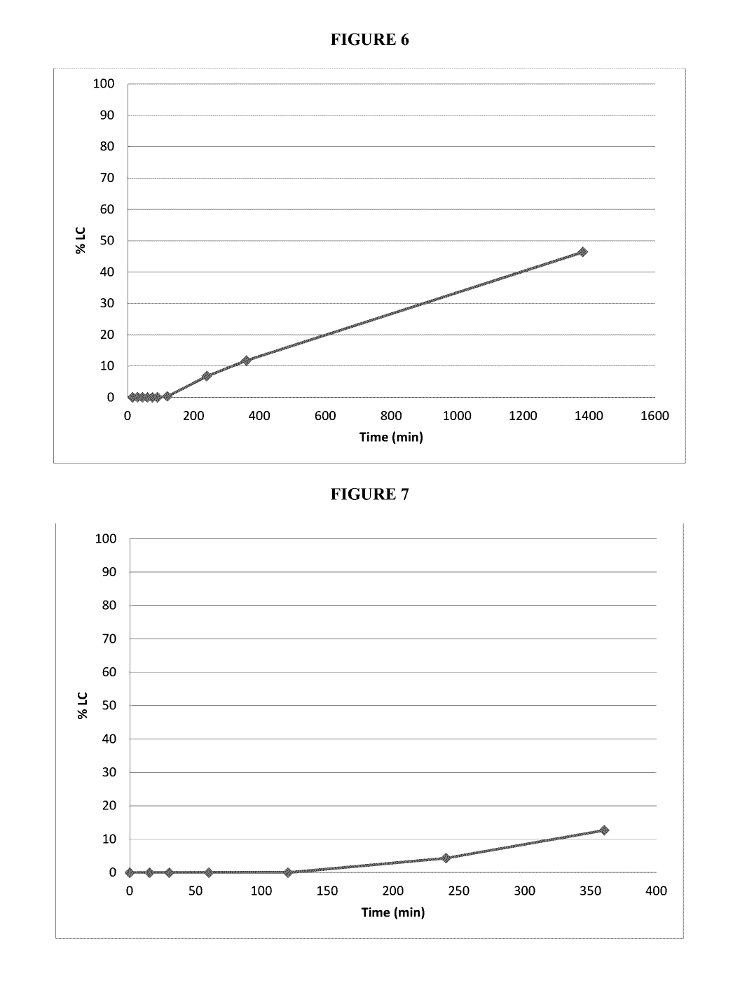

Subsequent experimental tests were run to obtain the release profiles of these capsules. FIG. 6 shows the release profile of the coated capsules according to this example embodiment after 1 month's storage at room temperature. FIG. 7 shows the release profile of the coated capsules according to this example embodiment after 15 months' storage at room temperature.

As seen in FIGS. 6 and 7, the coated softgel capsules according to this example embodiment enable zero-order or close to zero-order release of the liquid/semi-solid fill from the capsules without the use of semi-solid or solid lipid-based matrices.

### Example 3

Gelatin-based softgel capsule formulations containing 200 mg Ibuprofen were prepared according to the composition set forth in Table 1. The capsules were subsequently coated with the film coating composition set forth in Table 4 in order to achieve extended release softgel capsules.

TABLE 4
Coating Composition.

| Ingredient | % w/w | mg/capsule |
|---|---|---|
| Ethyl acrylate and methyl methacrylate copolymer | 71.99 | 41 |
| (Eudragit NE 30D) | | |
| Hypromellose (Pharmacoat 603) | 20.01 | 11 |
| PlasACRYL T20 (water, glyceryl monostearate, | 8.00 | 5 |
| polysorbate 80, triethyl citrate) | | |
| DI Water | N/A | N/A |
| Total | 100.00 | 57 |

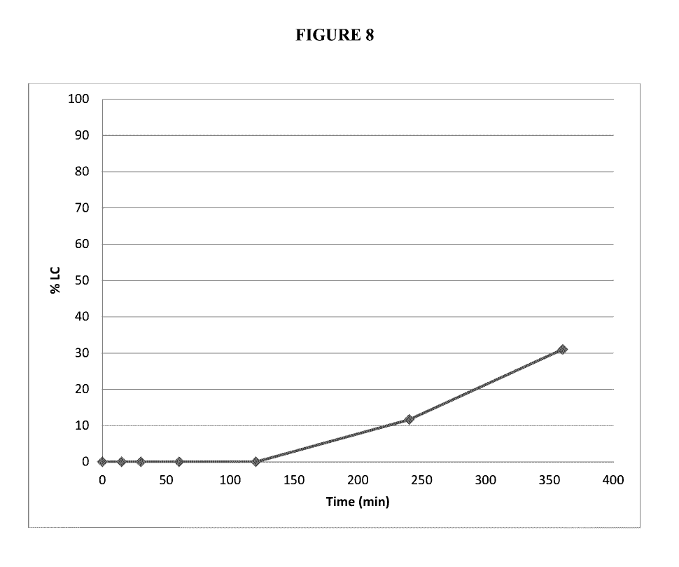

Subsequent experimental tests were run to obtain the release profiles of these capsules. FIG. 8 shows the release profile of the coated capsules according to this example embodiment (10.7-11.5% weight gain).

As seen in FIG. 8, the coated softgel capsules according to this example embodiment enable zero-order or close to zero-order release of the liquid/semi-solid fill from the capsules without the use of semi-solid or solid lipid-based matrices.

### Example 4

Gelatin-based softgel capsule formulations containing 200 mg Ibuprofen were prepared according to the composition set forth in Table 1. The capsules were subsequently coated with the film coating composition set forth in Table 5 in order to achieve extended release softgel capsules.

TABLE 5
Coating Composition.

| Ingredient | % w/w | mg/capsule |
|---|---|---|
| Ethyl acrylate and methyl methacrylate copolymer | 71.99 | 77 |
| (Eudragit NE 30D) | | |
| Hypromellose (Pharmacoat 603) | 20.01 | 21 |
| PlasACRYL T20 (water, glyceryl monostearate, | 8.00 | 9 |
| polysorbate 80, triethyl citrate) | | |
| DI Water | N/A | N/A |
| Total | 100.00 | 107 |

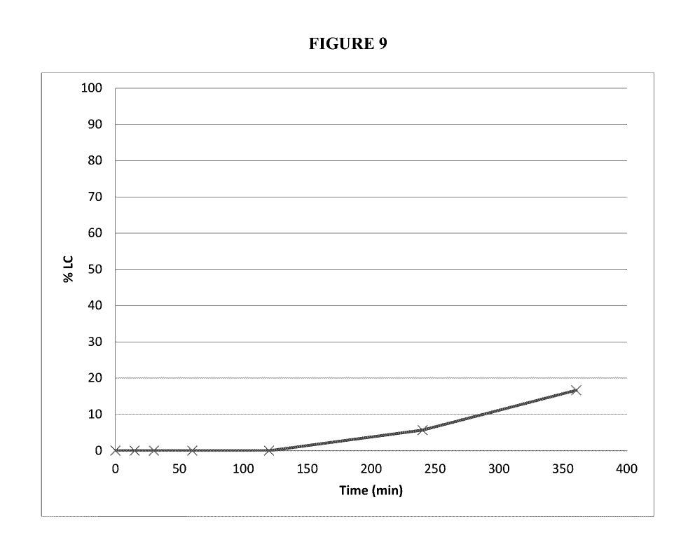

Subsequent experimental tests were run to obtain the release profiles of these capsules. FIG. 9 shows the release profile of the coated capsules according to this example embodiment (20% weight gain). As seen in FIG. 9, the coated softgel capsules according to this example embodiment enable zero-order or close to zero-order release of the liquid/semi-solid fill from the capsules without the use of semi-solid or solid lipid-based matrices.

### Example 5

Gelatin-based softgel capsule formulations containing 200 mg Ibuprofen were prepared according to the composition set forth in Table 1. The capsules were subsequently coated with the film coating composition set forth in Table 6 in order to achieve extended release softgel capsules.

TABLE 6
Coating Composition.

| Ingredient | % w/w | mg/capsule |
|---|---|---|
| Ethylcellulose dispersion (Aquacoat ECD 30) | 62.50 | 85 |
| Polyvinyl alcohol/polyethylene glycol co-polymer | 12.50 | 17 |
| (Kollicoat IR) | | |

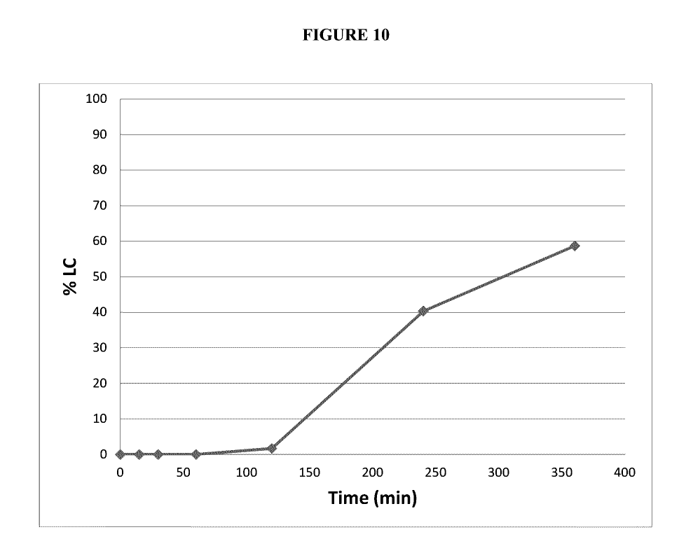

Subsequent experimental tests were run to obtain the release profiles of these capsules. FIG. 10 shows the release profile of the coated capsules according to this example embodiment (25.3-26.1% weight gain). As seen in FIG. 10, the coated softgel capsules according to this example embodiment enable zero-order or close to zero-order release of the liquid/semi-solid fill from the capsules without the use of semi-solid or solid lipid-based matrices.

### Example 6

Gelatin-based softgel capsule formulations containing 200 mg Ibuprofen were prepared according to the composition set forth in Table 1. The capsules were subsequently coated with the film coating composition set forth in Table 7 in order to achieve extended release softgel capsules.

TABLE 7
Coating Composition.

| Ingredient | % w/w | mg/capsule |
|---|---|---|
| Ethylcellulose dispersion (Aquacoat ECD 30) | 62.50 | 133 |
| Polyvinyl alcohol/polyethylene glycol co-polymer | 12.50 | 27 |
| (Kollicoat IR) | | |
| Triethyl citrate | 25.00 | 54 |
| DI Water | N/A | N/A |
| Total | 100.00 | 215 |

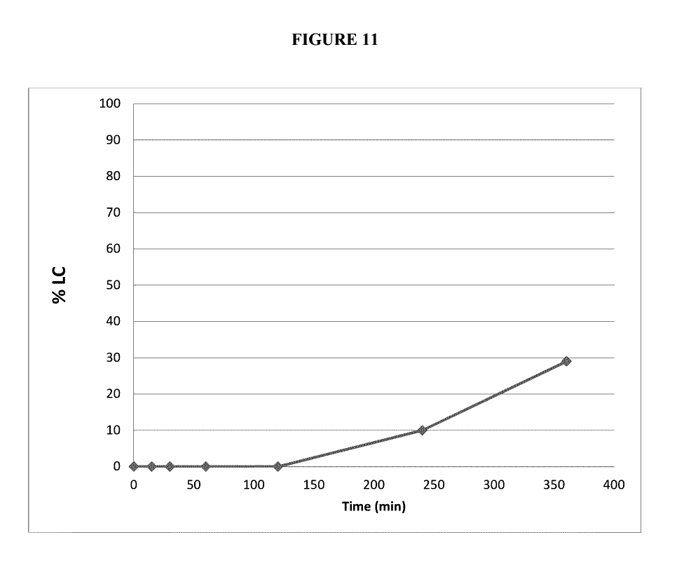

Subsequent experimental tests were run to obtain the release profiles of these capsules. FIG. 11 shows the release profile of the coated capsules according to this example embodiment (39.6-40.3% weight gain). As seen in FIGS. 11, the coated softgel capsules according to this example embodiment enable zero-order or close to zero-order release of the liquid/semi-solid fill from the capsules without the use of semi-solid or solid lipid-based matrices.

### Example 7

Gelatin-based softgel capsule formulations containing 180 mg Fexofenadine HCl were prepared according to the composition set forth in Table 8.

TABLE 8
Composition of Fexofenadine HCl 180 mg Softgel Capsule

| | % w/w | mg/capsule |
|---|---|---|
| Fexofenadine HCl | 11.986 | 180.00 |
| Polyethylene Glycol 300 | 54.204 | 814.00 |
| Acetic Acid | 0.533 | 8.00 |
| Povidone K-90 | 1.332 | 20.00 |
| Gelatin | 19.415 | 291.56 |
| Sorbitol, special | 5.579 | 83.77 |
| Glycerin | 6.940 | 104.22 |
| FD&C Blue #1 | 0.001 | 0.01 |
| D&C Red #33 | 0.011 | 0.16 |
| Total | 100.000 | 1501.73 |

The capsules were subsequently coated with the film coating composition set forth in Table 9 in order to achieve extended release softgel capsules.

TABLE 9
Coating Composition.

| Ingredient | % w/w | mg/capsule |
|---|---|---|
| Ethyl acrylate and methyl methacrylate | 72.01 | 93 |
| copolymer (Eudragit NE 30D) | | |
| Hypromellose (Pharmacoat 603) | 19.99 | 26 |
| PlasACRYL T20 (water, glyceryl | 8.00 | 10 |
| monostearate, polysorbate 80, triethyl | | |
| citrate) | | |
| DI Water | N/A | N/A |
| Total | 100.00 | 129 |

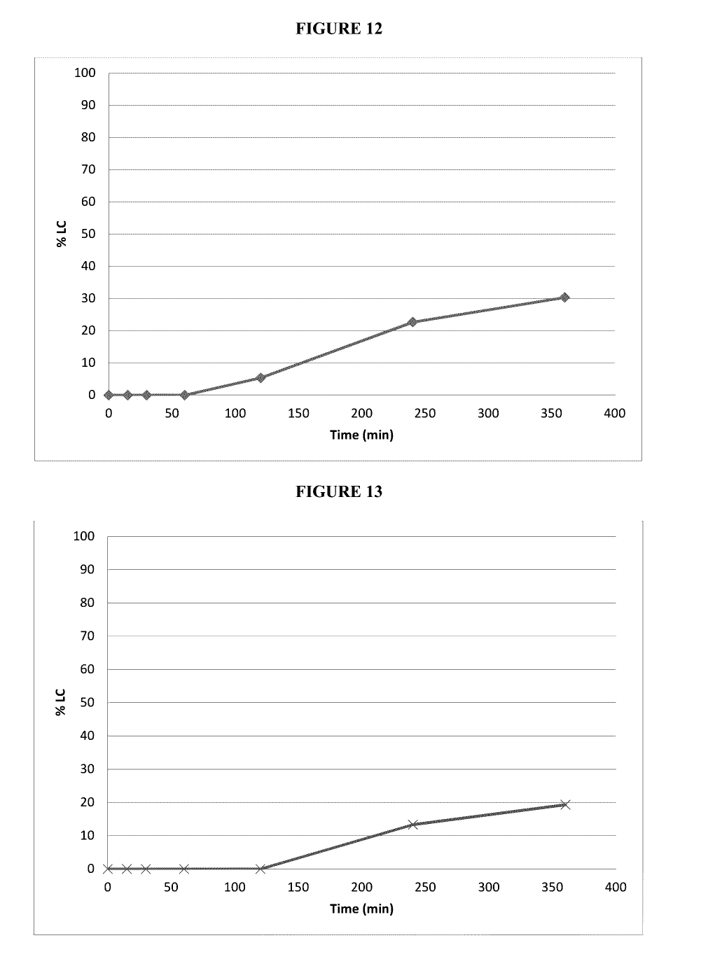

Subsequent experimental tests were run to obtain the release profiles of these capsules. FIGS. 12 and 13 show the release profile of the coated capsules according to this example embodiment (8.2-9.0% weight gain and 19.0-19.9% weight gain, respectively). As seen in FIGS. 12 and 13, the coated softgel capsules according to this example embodiment enable zero-order or close to zero-order release of the liquid/semi-solid fill from the capsules without the use of semi-solid or solid lipid-based matrices.

### Comparative Example 7

Gelatin-based softgel capsule formulations containing 180 mg Fexofenadine HCl were prepared according to the same composition in Example 7 as set forth in Table 8. However, the capsules in this comparative example remained uncoated.

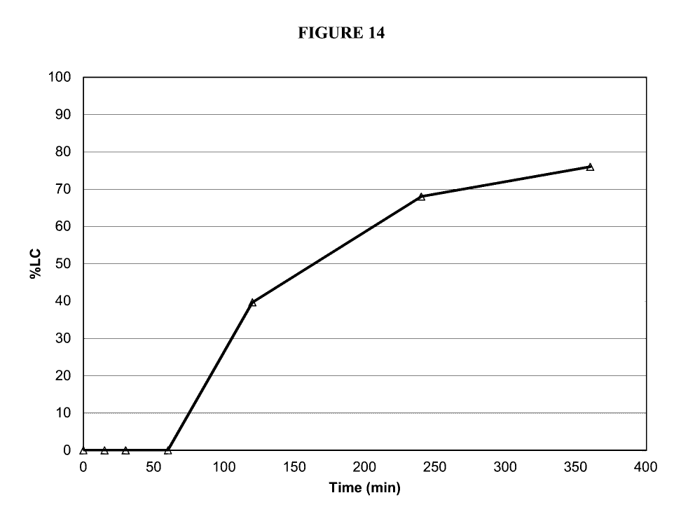

FIG. 14 shows that an uncoated softgel capsule of the composition set forth in Table 1, which is representative of softgel capsules in the state of the art, exhibits immediate-release of the fill material. In contrast, FIGS. 12 and 13 show that an example embodiment of the present invention enables zero-order or close to zero-order release from softgel capsules.

Numerous alterations, modifications, and variations of the preferred embodiments disclosed herein will be apparent to those skilled in the art, and they are all anticipated and contemplated to be within the spirit and scope of the claimed invention. For example, although specific embodiments have been described in detail, those with skill in the art will understand that the preceding embodiments and variations can be modified to incorporate various types of substitute, additional, or alternative materials. Accordingly, even though only few variations of the present invention are described herein, it is to be understood that the practice of such additional modifications and variations and the equivalents thereof, are within the spirit and scope of the invention as defined in the following claims. All patent applications, patents, and other publications cited herein are incorporated by reference in their entirety.

What is claimed is:

1. An extended release oral solid dosage form comprising:
(a) a fill material, said fill material comprising a liquid or semi-solid fill material containing at least one pharmaceutically active ingredient;
(b) a capsule containing the fill material, said capsule comprising a softgel capsule or a hard-shell capsule; and
(c) a coating surrounding the capsule, said coating comprising (1) a water-insoluble polymer and (2) a pore former.

2. The extended release oral solid dosage form of claim 1, wherein the water-insoluble polymer is a pH-independent polymer having low solubility in a pH range of about 1-8.

3. The extended release oral solid dosage form of claim 2, wherein the water-insoluble polymer is selected from the group consisting of ethylcellulose, ethyl acrylate and methyl methacrylate copolymer, polyvinyl acetate, cellulose acetate and combinations thereof.

4. The extended release oral solid dosage form of claim 2, wherein the water-insoluble polymer is present in the coating in an amount of about 1% to about 30% of the total dry polymer weight.

5. The extended release oral solid dosage form of claim 1, wherein the pore former is a water-soluble, pH-independent pore former.

6. The extended release oral solid dosage form of claim 5, wherein the pore former is selected from the group consisting of hypromellose, hydroxypropyl cellulose, hydroxyethyl cellulose, methyl cellulose, polyvinyl alcohol polyethylene glycol graft copolymer, povidone, sucrose, water-soluble sodium and potassium salts, gelatin, cyclodextrins, copovidone, dextrates, dextrose, lactitol, mannitol, erythritol, fructose, galactose, lactose, hydroxyethyl methylcellulose, maltodextrin, maltose, sorbitol, propylene glycol, xylitol, tagatose, trehalose, polyethylene glycols, poloxamers, polydextrose, polyvinyl alcohol, and combinations thereof.

7. The extended release oral solid dosage form of claim 5, wherein the pore former is present in the coating in an amount of about 1% to about 50% of the total dry polymer weight.

8. The extended release oral solid dosage form of claim 1, wherein the coating further comprises a plasticizer.

9. The extended release oral solid dosage form of claim 8, wherein the plasticizer is selected from the group consisting of triethyl citrate, tributyl citrate, acetyltriethyl citrate, acetyltributyl citrate, triacetin, propylene glycol, poloxamer, polyethylene glycols, dibutyl sebacate, butyl stearate, dibutyl phthalate, diethyl phthalate, dimethyl phthalate, and combinations thereof.

10. The extended release oral solid dosage form of claim 1, wherein a plasticizer is present in the coating in an amount of about 0% to about 60% of the total dry polymer weight.

11. The extended release oral solid dosage form of claim 1, wherein the coating further comprises a surfactant, an anti-foaming agent, a detackifying agent, or a combination thereof.

12. The extended release oral solid dosage form of claim 1, wherein the dosage form comprises a further coating applied underneath the coating as a sub-coat or on the surface of the coating as a top coat.

13. The extended release oral solid dosage form of claim 1, wherein the release profile remains substantially unchanged over the dosage form's shelf life.

14. The extended release oral solid dosage form of claim 1, wherein the fill material is an immediate-release fill material.

15. The extended release oral solid dosage form of claim 1, wherein the fill material is a hydrophilic fill material.

16. A process of preparing an extended release oral solid dosage form comprising the steps of:
(a) preparing a fill material, said fill material comprising a liquid or semi-solid fill material containing at least one pharmaceutically active ingredient;
(b) encapsulating the fill material of step (a) with a capsule, said capsule comprising a softgel capsule or a hard-shell capsule; and
(c) applying a coating onto the surface of the capsule, said coating comprising (1) a water-insoluble polymer and (2) a pore former.

17. The process of claim 16, wherein the water-insoluble polymer and the pore former are dissolved or dispersed in aqueous media.

18. The process of claim 16, wherein the water-insoluble polymer and the pore former are dissolved in a solvent or a mixture of solvents.

19. The process of claim 16, wherein step (c) comprises spraying the coating onto the surface of the capsule in a perforated coating pan, a semi-perforated coating pan, a non-perforated coating pan, a sugar coating pan or a fluid bed coater.

20. The process of claim 16, wherein step (c) is a batch process.

21. The process of claim 16, wherein step (c) is a continuous process.
